(19) United States
(12) Patent Application Publication
(10) Pub. No.: US 2005/0084472 A1
(43) Pub. Date: Apr. 21, 2005

(54) Triple emulsion cosmetic/dermatological product

(75) Inventors: Antoine Gedouin, Saint-Coulomb (FR); Romuald Vallee, Saint-Meloir-des-Ondes (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Codif International SAS, Saint-Malo Cedex (FR)

(21) Appl. No.: 10/497,913

(22) PCT Filed: Nov. 28, 2002
(86) PCT No.: PCT/FR02/04105

(30) Foreign Application Priority Data
Dec. 7, 2001 (FR) 01/16174

(51) Int. Cl. A61K 7/06; A61K 31/522; A61K 35/80

(52) U.S. Cl. 424/74; 424/195.17; 514/263.31

(57) ABSTRACT

The invention concerns a cosmetic/dermatological product comprising a triple water-in-oil-in-water (W/O/W) emulsion including an outer aqueous phase, an intermediate oily phase and an inner aqueous phase. Said inner aqueous phase and/or said oily phase contains at least an active principle promoting lipolysis and/or an active principle inhibiting lipogenesis and said intermediate oily phase consists of oleosomes. The invention also concerns a method for obtaining as well as a method for using such a cosmetic/dermatological product.

The present invention concerns a cosmetic or dermatological product to prevent and/or act on cellulite. The invention also concerns a process of manufacturing this product as well as a use of the latter by application to the skin.

A large number of cosmetic products having a slimming action are available on the market. Some of these products are in the form of an emulsion.

In the state of the art, a type of emulsion, called triple or multiple emulsion, is also known. A triple emulsion comprises three phases: an inner phase dispersed in the form of droplets in an intermediate phase, itself dispersed in the form of droplets in an outer phase. It is known in particular how to make triple water-in-oil-in-water emulsions (conventionally noted W/O/W) by preparing in a first step a simple water-in-oil emulsion (W/O) and by dispersing, in a second step, this emulsion in water.

A method for obtaining a triple emulsion W/O/W in a single step has been proposed in the document WO-A-98 55 088. According to this method, a combination of hydrophilic and hydrophobic surfactants is dissolved in an oily phase, the oily phase is subsequently added to into the aqueous phase, then dispersed in a single step by agitation. The formation of oil droplets surrounded by a layer of liquid crystals is observed. These droplets, also called oleosomes, contain an emulsion W/O. In the end, a triple emulsion W/O/W is obtained. In this type of triple emulsion, it will be noted that the composition of the outer aqueous phase is identical to that of the inner aqueous phase.

The present invention results from the discovery according to which a triple emulsion obtained by said process in one step and comprising in its aqueous phase and/or in its oily phase slimming active principles is, in a surprising way, substantially more efficient than a conventional simple emulsion.

More precisely, the present invention is defined by a cosmetic product comprising a triple water-in-oil-in-water emulsion (W/O/W) comprising an outer aqueous phase, an intermediate oily phase and an inner aqueous phase, said inner aqueous phase and/or said oily phase containing at least one active principle promoting lipolysis and/or inhibiting lipogenesis and said intermediate oily phase being constituted by oleosomes.

The invention is also defined by a dermatological product comprising a triple water-in-oil-in-water emulsion (W/O/W) comprising an outer aqueous phase, an intermediate oily phase and an inner aqueous phase, said inner aqueous phase and/or at least said oily phase containing at least one active principle promoting lipolysis and/or inhibiting lipogenesis and said intermediate oily phase being constituted by oleosomes.

The invention further concerns a process of manufacturing said cosmetic or dermatological product. This process comprises a first step of preparing, in a separate manner, two phases, aqueous and oily, said oily phase containing hydrophilic and hydrophobic surfactants and said aqueous phase and/or the oily phase containing at least one slimming active principle, a second step of incorporating the oily phase to the aqueous phase and a third step of agitating the mixture thus formed until obtaining a triple emulsion W/O/W.

Finally, the invention concerns a use of said cosmetic product, so as to reduce the weight excess of a human body.

The preparation of the cosmetic/dermatological product according to the invention has been carried out under the following conditions.

For the preparation of the intermediate oily phase of the triple emulsion, a mixture of a highly polar oil (PPG-15-stearyl ether) with another oil of any type has been advantageously used. The addition of the components has been systematically made in the direction oil towards water. The dispersion has been made by agitation.

According to an experimental embodiment, the triple emulsion W/O/W is obtained by preparing separately each of the oily and aqueous phases.

The composition of the oily phase is as follows:
2% steareth-21
3% steareth-2
0.5% PEG-30 Dipolyhydroxystearate (PEG-Polyethyleneglycol)
5% PPG-15 stearyl ether (PPG =polypropyleneglycol)
2% jojoba oil
1% cetylic alcohol
1.5% stearic palmitic acid
1% dimethicone
1% phenoxyethanol+methylparaben+ethylparaben+isobutylparaben+butylparaben+propylparaben (paraben=parahydroxybenzoate)
2% sweet almond oil

On the other hand, the composition of the aqueous phase is as follows:
QSP demineralized water
Colorings
4% propyleneglycol
0.2% caffeine anhydrous powder
1% Phyco R75 (algae extract (laminaria))
5% Rhodofiltrat Palmaria HG (alga)
1.3% acrylamide/sodium acryloyldimethyltaurate+isohexadecane+polysorbate 80 copolymer

Each of the two phases is then heated to 80° C.

The aqueous phase is dispersed under high agitation (3000 tr/min during 5 min), then the oily phase is added to the aqueous phase under vacuum (−0.3 Bar) and under high agitation (60 tr/min and counter agitation 180 tr/min).

The agitation is maintained for 15 min, then the mixture is left to cool down to a temperature of about 53-55° C. Once this temperature is reached, 0.1% extract of Ondontella Aurita, perfume, and Thiethanolamine are added to the mixture thus obtained. Agitation is carried out again during 15 minutes before letting cool to obtain a temperature of 25-30° C.

The cosmetic composition obtained comprises three phases: an inner aqueous phase, an intermediate oily phase and an outer aqueous phase. As has been said, the inner and outer aqueous phases have the same composition. The hydrophilic active principles incorporated within the starting inner aqueous phase (caffeine, Phyco R75, Rhodofiltrat Palmaria), are found after formation of the emulsion within the droplets of the inner aqueous phase as well as in the outer aqueous phase. The lipophilic active principles (here, the extract of Odontella Aurita) added to the mixture are found, for their part, in the intermediate oily phase.

The intermediate oily phase is in the form of oily droplets surrounded by liquid crystal lamellae, in other words, oleosomes.

According to a preferred embodiment, the active principles used are algae extracts. However, other active principles having slimming properties such as from xanthic bases such as caffeine can be envisioned, in some cases, in combination with said algae extracts. Thus, in the example above, the active principles are: Phyco R75, Rhodofiltrat Palmaria HG, caffeine and an extract of Odontella Aurita.

As will be seen below, it has been shown through tests that the action of these active principles is substantially more pronounced in the case of a triple emulsion than in the case of a simple emulsion.

The cosmetic/dermatological product according to the invention is applied onto the skin of the part of the body to be treated. The action of said product is attributed to the following mechanism. The droplets composing the intermediate oily phase of the triple emulsion or oleosomes migrate in the direction of the adipocytes located in the hypodermis. These oleosomes have an average size about equal to 15 μm. Having come into contact with the adipocytes, the oleosomes coalesce with the adipocytes and release within them the active principles that they contain.

The W/O/W emulsion obtained by the process in a single step makes it possible to reach the hypodermis more easily than a simple emulsion or even than a normal triple emulsion and enables a targeted release of the active principles.

Depending on the type of active principle contained in the aqueous phase and/or the oily phase, the action on the adipocytes will not be the same. Some active principles degrade the fat present in the adipocytes, this is the phenomenon called lipolysis. Others act on the adipocytes by preventing the formation of fat (triglycerides) within the adipocytes, this is the phenomenon called inhibition of the lipogenesis. Depending on the case, a reduction of cellulite or a prevention of its worsening is obtained.

Among the active principles promoting lipolysis, caffeine and: Phyco R75, Rhodofiltrat Palmaria HG, will be preferentially used. Among the active principles inhibiting lipogenesis, an extract of Odontella Aurita will be preferentially used.

A series of tests has made it possible to show the increased efficacy of the triple emulsion used in the cosmetic/dermatological product according to the invention, first with respect to a simple triple emulsion, hereinafter called normal emulsion, but also with respect to a conventional triple emulsion, hereinafter called placebo 2.

The tests were performed on two groups each made up of twenty volunteers and the results were obtained by measuring the perimeter of the ankles, that of the thighs, as well as the thickness of the hypodermis by ultrasound.

The first consisted in comparing a simple emulsion containing active principles (so-called normal emulsion) with a placebo simple emulsion (placebo 1).

The second test consisted in comparing a zone treated by a placebo triple emulsion (placebo 2) to a non-treated zone.

And the third test consisted in comparing a zone treated with the triple emulsion according to the invention with a non-treated zone.

The objective of these tests is to evaluate the efficacy of the triple emulsion according to the invention, as compared to a simple emulsion containing the same active principles, and as compared to a conventional triple emulsion.

For each of these tests, a test is performed and the values obtained during these tests are compared to each other.

The test used is the test t of Student on matched values. The principle of the test consists in setting a null hypothesis (H0) of an absence of any difference between the average effects of the two treatments (d=0) and an alternative hypothesis HI (the research hypothesis) of a difference between the two treatments d<>0). The probability p of observing a discrepancy between the two treatments at least as large as that which was observed if the null hypothesis is true is then determined.

If p <5%, the null hypothesis is rejected. The alternative hypothesis H1 of a significant difference between the two treatments is then accepted.

If p >5%, H0 is accepted. The data did not make it possible to show a significant difference between the two treatments.

In the case of non-normality of the differences, a Wilcoxon test is performed.

The principle of the test being defined, measurements of the thickness of the adipose tissue are performed directly on the ultrasound negatives in the area of the front, lateral and back faces of the thighs. The thicknesses are given in millimeters.

The first test consisted in comparing the averages and the standard deviations with respect to the averages (SDA) obtained on the average of three zones of the thighs (front, lateral, and back faces of the thighs) after 56 days of application twice daily of, on the one hand, a normal emulsion, and on the other hand, a placebo 1 (Table 1).

The results are as follows:

TABLE 1

| | |
|---|---|
| Normal emulsion | Placebo 1 |

| | J0 | J56 | J0 | J56 |
|---|---|---|---|---|
| Average | 14.2 | 14.0 | 14.0 | 13.9 |
| SDA | 0.6 | 0.6 | 0.6 | 0.7 |

| | | |
|---|---|---|
| Δ(J56-J0) | −0.1 ± 0.3 | −0.1 ± 0.4 |
| Δ%(J56-J0) | −1% | −1% |

| | |
|---|---|
| Δ Emulsion/Placebo | 0.0 ± 0.4 |
| | p = 0.942 |
| Δ% Emulsion/Placebo | 0% |

As indicated by the Student test, no significant difference is observed between the thigh treated by the normal emulsion and the thigh treated by the placebo 1.

The second test consisted in evaluating a placebo 2 alone, so as to show, on the one hand, the average values of the thickness of the hypodermis of the volunteers using the product during the test (Table 2), and on the other hand, the average variations in the thickness of the hypodermis of the volunteers using the placebo 2 (Table 3).

Thus, the results obtained without the placebo (NTZ) and with the placebo (TZ) are compared after 28 and 56 days.

The results are as follows:

TABLE 2

| | Zone treated | | | Zone not treated | | |
|---|---|---|---|---|---|---|
| | by the placebo (TZ) | | | by the placebo (NTZ) | | |
| | (in mm) | | | (in mm) | | |
| | J0 | J28 | J56 | J0 | J28 | J56 |
| Average | 35.8 | 35.7 | 32.6 | 35.3 | 35.7 | 33.0 |
| SDA | 2.2 | 2.3 | 2.3 | 2.5 | 2.5 | 2.6 |

TABLE 3

| | |
|---|---|
| Δ TZ/NTZ | Δ% TZ/NTZ |

| | J28-J0 | J56-J0 | J28-J0 | J56-J0 |
|---|---|---|---|---|
| Average | −0.5 | −1.6 | −0.9 | −2.7 |
| SDA | 0.8 | 1.3 | 2.2 | 4.1 |
| Student p | 0.501 | 0.224 | | |

It is observed that after 28 and 56 days of using placebo 2 twice daily, the thickness of the hypodermis is not reduced significantly for the treated thigh, as compared to the non-treated thigh.

The third test consisted in comparing the variations of results obtained between a zone treated by the multiple emulsion according to the invention and a non-treated zone.

These results are summarized, on the one hand, in Table 4 giving average values of the thickness of the hypodermis in the zone treated with the triple emulsion, as compared to the non-treated zone at 28 and 56 days, and on the other hand, in Table 5 giving the average variations of the thickness of the hypodermis between the zone treated by the triple emulsion and the non-treated zone.

TABLE 4

| | Treated zone (TZ) | | | Non-treated zone (NTZ) | | |
|---|---|---|---|---|---|---|
| | (in mm) | | | (in mm) | | |
| | J0 | J28 | J56 | J0 | J28 | J56 |
| Average | 30.5 | 28.2 | 29.2 | 25.9 | 27.3 | 29.0 |
| SDA | 2.1 | 1.8 | 2.2 | 2.0 | 1.8 | 2.1 |

TABLE 5

| | |
|---|---|
| Δ treated zone/ | Δ% treated zone/ |
| non-treated zone | non-treated zone |
| (in mm) | (in %) |

| | J28-J0 | J56-J0 | J28-J0 | J56-J0 |
|---|---|---|---|---|
| Average | −2.6 | −4.6 | −6.8 | −10.8 |
| (n = 20) | | | | |
| SDA | 1.2 | 1.1 | 4.0 | 4.4 |
| p | 0.043 | <0.001 | | |

It is then observed that after 28 and 56 days of using twice daily the triple emulsion according to the invention, the thickness of the hypodermis is reduced significantly on the treated thigh, as compared to the non-treated thigh. This is confirmed by the Student test which gives at 56 days a significant variation p <0.001.

Thus, the experimental results show an efficacy of the triple emulsion according to the invention considerably superior to that of the simple emulsion and considerably superior to that of a conventional triple emulsion.

1. Cosmetic product comprising a triple water-in-oil-in-water emulsion (W/O/W) comprising an outer aqueous phase, an intermediate oily phase and an inner aqueous phase, wherein said inner aqueous phase and/or said oily phase contains at least one active principle promoting lipolysis and/or active pinciple inhibiting lipogenesis, said intermediate oily phase being constituted by oleosomes.

2. Cosmetic product according to claim 1, wherein at least one active principle is constituted by extracts from algae.

3. Cosmetic product according to claim 1, wherein at least one active principle is a xanthic base, in particular caffeine.

4. Cosmetic product according to claim 1, wherein said intermediate oily phase contains an extract of Odontella Aurita as active principle inhibiting lipogenesis.

5. Dermatological product comprising a triple water-in-oil-in-water emulsion (W/O/W) comprising an outer aqueous phase, an intermediate oily phase and an inner aqueous phase, wherein at least said inner aqueous phase contains at least one active principle promoting lipolysis and/or at least said intermediate oily phase contains at least one active principle inhibiting lipogenesis, said intermediate oily phase being consistuted by oleosomes.

6. Dermatological product according to claim 5, wherein at least one active principle is constituted by algae extracts.

7. Dermatological product according to claim 5, wherein at least one active principle is a xanthic base, in particular caffeine.

8. Dermatological product according to claim 5, wherein said intermediate oily phase contains an extract of Odontella Aurita as active principle inhibiting lipogenesis.

9. Process of manufacturing the cosmetic product according to claim 1, comprising a first step of preparing the two phases, aqueous and oily, separately, said oily phase containing hydrophilic and hydrophobic surfactants, said aqueous phase and/or the oily phase containing at least one slimming active principle, a second step of incorporating the oily phase to the aqueous phase, and a third step of agitating the mixture thus formed until obtaining a triple emulsion W/O/W.

10. Process of manufacturing the dermatological product according to claim 5, comprising a first step of preparing the two phases, aqueous and oily, separately, said oily phase containing hydrophilic and hydrophobic surfactants, said aqueous phase containing at least one slimming principle, a second step of incorporating the oily phase to the aqueous phase, and a third step of agitating the mixture thus formed until obtaining a triple emulsion W/O/W.

11. Process of using the cosmetic product according to claim 1 comprising applying the product cutaneously on a human body so as to reduce a weight excess of the latter.

12. Cosmetic product according to claim 2, wherein at least one active principle is a xanthic base, in particular caffeine.

13. Cosmetic product according to claim 2, wherein said intermediate oily phase contains an extract of Odontella Aurita as active principle inhibiting lipogenesis.

14. Cosmetic product according to claim 3, wherein said intermediate oily phase contains an extract of Odontella Aurita as active principle inhibiting lipogenesis.

15. Cosmetic product according to claim 12, wherein said intermediate oily phase contains an extract of Odontella Aurita as active principle inhibiting lipogenesis.

16. Dermatological product according to claim 6, wherein at least one active principle is a xanthic base, in particular caffeine.

17. Dermatological product according to claim 6, wherein said intermediate oily phase contains an extract of Odontella Aurita as active principle inhibiting lipogenesis.

18. Dermatological product according to claim 7, wherein said intermediate oily phase contains an extract of Odontella Aurita as active principle inhibiting lipogenesis.

19. Dermatological product according to claim 16, wherein said intermediate oily phase contains an extract of Odontella Aurita as active principle inhibiting lipogenesis.
